(19) United States
(12) Patent Application Publication
(10) Pub. No.: US 2005/0233961 A1
(43) Pub. Date: Oct. 20, 2005

(54) Methods for treating cancer using vascular endothelial cell growth inhibitor VEGI-192A

(76) Inventors: Xinli Lin, Piedmont, CA (US); Luyuan Li, Pittsburgh, PA (US)

Correspondence Address: MORRISON & FOERSTER LLP, 755 PAGE MILL RD, PALO ALTO, CA, 94304-1018, US

(21) Appl. No.: 11/011,456

(22) Filed: Dec. 13, 2004

Related U.S. Application Data
(60) Provisional application No. 60/529,173, filed on Dec. 11, 2003.
(60) Provisional application No. 60/536,653, filed on Jan. 13, 2004.

(51) Int. Cl. A61K 38/18

(52) U.S. Cl. 514/12

(57) ABSTRACT

The invention features methods and compositions for treating cancer by administering an effective amount of a VEGI-192A polypeptide. In some embodiments, the cancer is lung cancer or breast cancer.

### CROSS-REFERENCE TO RELATED APPLICATIONS

This application claims the priority benefit of provisional patent applications U.S. Ser. No. 60/529,173, filed Dec. 11, 2003 and No. 60/536,653, filed Jan. 13, 2004, which are incorporated in their entirety by reference.

### STATEMENT REGARDING FEDERALLY SPONSORED RESEARCH OR DEVELOPMENT

This invention was made with U.S. Government support under National Institutes of Health grant N1H-HL060660; and N1H-CA102181. The U.S. Government may have certain rights in this invention.

### FIELD OF THE INVENTION

This invention relates to methods for treating cancer using vascular endothelial growth inhibitor VEGI-192A.

### BACKGROUND OF THE INVENTION

Vascular endothelial cell growth inhibitor (VEGI) is an endothelial cell-specific gene product. Four isoforms of human VEGI have been reported: The first form of VEGI discovered is 174 amino acids in length; two different forms of 192 amino acid residues and one of 251 amino acid residues are later discovered. See Zhai et al., Int. J. Cancer 82:131-136 (1999); Zhai et al., FASEB J. 13: 181-189 (1999); Chew et al., FASEB J. 16: 742-744 (2002); PCT WO03/039491; U.S. Pat. Appl. Pub. No. 2003/0170242. All isoforms are splicing variants arising from a common gene. The four isoforms differ in their N-terminal regions but share an identical core of 151 amino acids encoding the rest of the protein.

A comparison of the sequences of the four isoforms indicates that they share 20-30% identity with the tumor necrosis factor (TNF) superfamily of proteins. The roles of the different isoforms have not been clearly delineated and is undoubtedly subtle and complex—with both membrane bound and secreted forms of the molecule being reported. All of the evidence reported thus far seems to point to secreted forms of VEGI-174 inhibits tumor cell growth and initiates apoptosis. Hydrophobicity profiling of VEGI-174 implies that it is a typical type II transmembrane protein, with amino acids 29-1.74 constituting the extracellular domain. Full length VEGI-174 does not have any effect on tumor growth when overexpressed in cancer cells, nor does it inhibit endothelial cells when transfected into these cells. See Zhai et al., FASEB J. 13: 181-189 (1999); Chew et al., FASEB J. 16: 742-744 (2002). Several members of the TNF family, including TNF and Fas ligand have been shown to be cleaved from the membrane and function as soluble proteins. See Bjomberg et al., Scand J. Immunol. 42: 418-424 (1995); Kayagaki et al., J. Exp. Med. 182: 1777-83 (1995). This has not yet been demonstrated for VEGI-174. Nevertheless, an artificial recombinant secretory form of this VEGI-174 (s-VEGI) comprising only the extracellular domain of VEGI-174 and a secretion signal peptide derived from a secretory protein inhibited tumor growth when overexpressed in cancer cells. See Zhai et al., Int. J. Cancer 82:131-136 (1999); U.S. Pat. Appl. Pub. No. 2002/0111325. VEGI-251, the most abundant isoform, possesses a putative secretory signal peptide. Over-expression of VEGI-251 causes endothelial cell apoptosis and growth inhibition. PCT WO03/039491; U.S. Pat. Appl. Pub. No. 2003/0170242. Similarly, recombinant VEGI that contains the 151 amino acid core sequence shared by all forms of VEGI has been shown to initiate apoptosis and block tumor cell growth, albeit with low potency. See Wang, et al., Acta Biochimica et Biophysica Sinica 32(5): 485-489 (2000). It has been reported that recombinantly produced and refolded VEGI-192A inhibited proliferation of adult bovine aortic endothelial (ABAE) cells in vitro. PCT WO03/039491; U.S. Pat. Appl. Pub. No. 2003/0170242. Methods for refolding proteins have been reported in U.S. Pat. No. 6,583,268.

All patents, patent applications, and publications cited herein are hereby incorporated by reference in their entirety. It should be noted that reference to a publication in this Background section is not an admission that the publication constitutes prior art to the instant invention.

### BRIEF SUMMARY OF THE INVENTION

The present invention is based on the discovery that recombinantly produced VEGI-192A protein is effective in treating cancer, such as lung and breast cancer.

In one aspect, the invention is a method of treating cancer in an individual comprising administering an effective amount of a VEGI-192A polypeptide to the individual. In some embodiments, the cancer is lung or breast cancer.

In another aspect, the invention is a method of inhibiting tumor growth in an individual comprising administering to the individual an effective amount of a VEGI-192A polypeptide to the individual. In some embodiments, the cancer is lung or breast cancer.

In another aspect, the invention is a method of delaying progression of cancer in an individual comprising administering to the individual an effective amount of a VEGI-192A polypeptide to the individual. In some embodiments, the cancer is lung or breast cancer.

In another aspect, the invention is a method of delaying development of metastasis in an individual with cancer comprising administering an effective amount of a VEGI-192A polypeptide to the individual. In some embodiments, the cancer is lung or breast cancer.

In another aspect, the cancer being treated is advanced. In some embodiments, the advanced cancer is lung or breast cancer.

In another aspect, the invention is a pharmaceutical composition comprising a VEGI-192A polypeptide (in some embodiments, an effective amount of a VEGI-192A polypeptide) and a pharmaceutical acceptable excipient.

In another aspect, the invention provides a kit for use in any of the methods described herein. In some embodiments, the kit comprises a container, a composition comprising a VEGI-192A polypeptide in combination with a pharmaceutical acceptable carrier, and instructions for using the composition in any of the methods described herein.

In some embodiments of the invention, the VEGI-192A polypeptide is human VEGI-192A. in some embodiments, the VEGI-192A polypeptide comprises amino acid sequence of SEQ ID NO:1.

In some embodiments, the individual is a mammal. In some embodiments, the individual is a human.

### BRIEF DESCRIPTION OF THE SEVERAL VIEWS OF THE DRAWING(S)

FIG. 1 shows purification of refolded VEGI-192A (with His-Tag) and endothelial-cell (ABAE) growth arrest assay. FIG. 1A shows Sephacryl S-300 column chromatography. The column was equilibrated and run with 20 mM Tris, 0.2 M NaCl, 0.4 M urea, pH 8.0. On the top of the peaks, 1, 2, and 3 represent three pools of fractions. FIG. 1B shows nonreducing SDS PAGE analysis of chromatography fractions, pools 1, 2, and 3, shown in FIG. 1A. FIG. 1C shows that endothelial cell growth arrest assay of pool 3, indicating the 50% inhibition concentration (IC50) is 12 ng/ml (0.5 nM).

FIG. 2 shows affinity purification of VEGI-192A (with His-Tag). FIG. 2A shows Ni+-affinity column purification of refolded VEGI-192A. Arrow indicates VEGI-192A peak. FIG. 2B shows SDS-PAGE of pooled and dialyzed sample from FIG. 2A. 1, non-reduced; 2, reduced.

FIG. 3A shows inhibition of Lewis lung tumor growth by intraperitoneal (IP) or intratumoral (IT) injection of VEGI-192A (with His-Tag) prepared as described in the Examples. FIG. 3B shows increased survival time of the tumor-bearing mice after VEGI-192A injection.

FIG. 4 shows increased apoptotic endothelial cells and decrease of microvessel density in mice bearing human breast cancer xenograft tumors formed by MDA-MB-231 cancer cells after intratumoral injection with VEGI-192A (with His-Tag) prepared as described in the Examples. Panel A, untreated tumors fluorescent labeled for apoptotic cells; Panel B, untreated tumors immunostained for CD31 (an endothelial cell marker); Panel C, VEGI-192A treated tumor fluorescent labeled for apoptotic cells; Panel D, VEGI-192A treated tumors immunostained for CD31 (an endothelial cell marker).

FIG. 5 shows inhibition of Lewis lung cancer (LLC) in-take by VEGI-192A. LLC cells were inoculated on the back of C57B1 mice. VEGI-192A (5 mg/Kg) was intraperitoneally injection (IP) on the same day (Day 0) of LLC cell inoculation. Tumor volumes for both vehicle and VEGI-192A treated (referred as “VEGI” in the graph) were determined on Day 4.

FIG. 6 shows spleen weight of LLC inoculated mouse after VEGI-192A treatment. LLC inoculated mice were treated with VEGI-192A (5 mg/Kg) on Day 4 (when the tumors became palpable), Day 7, Day 8, Day 9, and Day 10. Spleens were retrieved and weighed on Day 11.

FIG. 7A shows mouse serum profile of cytokines after VEGI-192A treatment. Cytokine serum concentrations were expressed in pg/ml. Sera were collected on Day 11. Cytokine levels were determine using an antibody-conjugated luminescent assay kit (LINCOplex). FIG. 7B shows data of FIG. 7A expressed as fold change of serum cytokine levels in VEGI-192A treated animals as compared to vehicle treated. “Experimental” refers to VEGI-192A treated group. “Control” refers to vehicle treated group. “*” indicates statistical difference (t-test, alpha=0.05).

FIG. 8 shows comparison of cytokine profiles in mouse serum (data shown in FIG. 7B) to cytokine expression profiles for both proliferating and confluent (G0-synchronized) HUVEC. Proliferating or confluent HUVEC cells were treated with 500 ng/ml VEGI-192A. Cytokine expression profiles for HUVEC were determined using a cDNA microarray (from Fred Hutchinson Cancer Research Center).

FIG. 9 shows inhibition of the growth of newly implanted LLC tumors. LLC cells (1×10⁶ per injection per animal) were inoculated on the flank of a C57BL black mouse on Day 0. Recombinant VEGI-192A (20 mg/kg) was given on Days 5, 9, and 12 by intraperitoneal (IP) injection. Tumor volumes were measured immediately prior to VEGI-192A treatment. “No Rx” refers to vehicle treated group. “Rx VEGI IP” refers to VEGI-192A treated group. T-test, p<0.05 (treated groups n=9, untreated group n=9).

FIG. 10 shows inhibition of LLC tumor formation. LLC cells (1×10⁶ per injection per animal) were inoculated on the flank of a C57BL black mouse on Day 0. The animals were treated with recombinant VEGI-192A (20 mg/kg) (referred as “VEGI” in the graph) immediately following cancer cell inoculation. The treatment was repeated daily until Day 4 and the tumor volumes were determined on day 5. Asterisks: T-test, p<0.002 (untreated, n=5; treated, n=6).

FIG. 11 shows specific elimination of endothelial cells by VEGI-192A in LLC tumors. Tumors were retrieved at the end of the experiment (3 weeks) from VEGI-192A-treated animals and vehicle-treated controls and processed as described in Example 7. Sections of the tumors were subjected to fluorescent immunostaining. Endothelial cells and smooth muscle cells were identified with specific markers CD31 (red) and SMA (green), respectively. Panel A: Image of a typical tumor section from VEGI treated group; magnification, 200×. Panel B: Image of a typical tumor section from vehicle-treated group; magnification, 200×. Panel C: Quantitative analysis of red and green areas of the images of the tumors. White bars, CD31-positive endothelial cells. Black bars, SMA-positive smooth muscle cells. Asterisks, T-test, p<0.01 between vehicle and VEGI treated groups for CD31 staining (5 animals per group; 15 areas/section analyzed).

FIG. 12 shows presence of residual vascular structures. Sections of LLC tumors from VEGI-192A- or vehicle-treated animals were subjected to immunostaining in order to identify endothelial cells (CD31), smooth muscle cells (SMA) and blood vessel basement membrane (collagen IV). Panel A: Image of a typical vehicle-treated tumor section showing CD31-positive vessels (brown), magnification, 1000×. Panel B: Image of a typical VEGI-192A-treated tumor section showing lumen-like spaces with red blood cells but lacked CD31-positive endothelial cells; magnification, 1000×. Panel C and C′ (inset): Images of typical VEGI-192A-treated tumor sections with CD31 (green) and collagen IV (red) double-staining; notice the lack of CD31+endothelial cells in the lumen-like space lined by collagen IV demonstrated in C′. Panel D and D′ (inset): Images of typical sections of VEGI-treated tumors with SMA (green) and collagen IV (red) staining; notice the presence of smooth muscle cells in the inner boarders of the lumen-like structures. Panel E and E′ (inset): Images of typical vehicle-treated tumor sections with CD31 (green) and collagen IV (red) staining; notice the presence of endothelial cells (CD31+) in the vessel walls. Panel F and F′ (inset): Images of typical vehicle-treated tumor sections with SMA (green) and collagen IV (red) staining; notice the presence of smooth muscle cells in the vessel walls. Blue staining, cell nuclei. Magnification for C, D, E, and F: 200×. Magnification for C′, D′, E′ and F′: 1000×.

### DETAILED DESCRIPTION OF THE INVENTION

The present invention is based on the discovery that administration of a therapeutically effective amount of VEGI-192A protein may be used to treat cancer including late stage (advanced) cancer, for example, lung and breast cancer.

I. General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

II. Definitions

An “effective amount” of a drug, or pharmaceutical composition is an amount sufficient to effect beneficial or desired results including clinical results such as shrinking the size of the tumor (in the cancer context, for example, breast or lung cancer), retardation of cancerous cell growth, decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival of individuals. An effective amount can be administered in one or more administrations. For purposes of this invention, an effective amount of drug, compound, or pharmaceutical composition is an amount sufficient to reduce the proliferation of (or destroy) cancerous cells and to reduce and/or delay the development, or growth, of metastases of cancerous cells, either directly or indirectly. As is understood in the cancer clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality, such as administration of VEGI-192A and other anti-cancer drug. As such, “in conjunction with” refers to administration of one treatment modality before, during or after administration of the other treatment modality to the individual.

As used herein, “treatment” or “treating” is an approach for obtaining beneficial or desired results including and preferably clinical results. For purposes of this invention, beneficial or desired clinical results include, but are not limited to, one or more of the following: reducing the growth of (or destroying) cancerous cells, reducing metastasis of cancerous cells found in cancers, shrinking the size of the tumor, decreasing symptoms resulting from the disease, increasing the quality of life of those suffering from the disease, decreasing the dose of other medications required to treat the disease, delaying the progression of the disease, and/or prolonging survival of individuals.

As used herein, “delaying development of metastasis” means to defer, hinder, slow, retard, stabilize, and/or postpone development of metastasis. This delay can be of varying lengths of time, depending on the history of the cancer and/or individual being treated. As is evident to one skilled in the art, a sufficient or significant delay can, in effect, encompass prevention, in that the individual does not develop the metastasis.

An “individual” is a vertebrate, preferably a mammal, more preferably a human. Mammals include, but are not limited to, farm animals, sport animals, pets (such as cats, dogs, horses), primates, mice and rats.

As used herein, “agent” refers to a biological, pharmaceutical, or chemical compound. Non-limiting examples include simple or complex organic or inorganic molecule, a peptide, a protein, an oligonucleotide, an antibody, an antibody derivative, or antibody fragment. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like.

As used herein, a “therapeutic agent” means any agent useful for therapy (here, generally in the cancer context) including anti-tumor drugs, toxins or cytotoxins, cytotoxin agents, and radioactive agents.

It should be noted that, as used herein, the singular form “a”, “an”, and “the” includes plural references unless indicated otherwise.

III. Methods of Using VEGI-192A for Therapeutic Purposes

The invention provides a method of treating cancer in an individual by administering an effective amount of a VEGI-192A to the individual. In some embodiments, the cancer is lung or breast cancer.

The invention also provides a method of inhibiting tumor growth (such as lung or breast cancer) in an individual by administering to the individual an effective amount of a VEGI-192A to the individual.

The invention also provides a method of delaying progression of cancer (such as lung or breast cancer) in an individual by administering to the individual an effective amount of a VEGI-192A to the individual.

The invention also provides a method of delaying development of metastasis in an individual with cancer (such as lung or breast cancer) by administering an effective amount of a VEGI-192A to the individual.

The invention also provides a method of inhibiting growth and/or proliferation of vascular endothelial cells in an individual with cancer (such as lung or breast cancer) by administering an effective amount of a VEGI-192A to the individual. The invention also provides a method of inhibiting angiogenesis in an individual with cancer (such as lung or breast cancer) by administering an effective amount of a VEGI-192A to the individual.

The cancer being treated may be advanced. In some embodiments, the advanced cancer is lung or breast cancer.

Various formulations of VEGI-192A may be used for administration. In some embodiments, VEGI-192A may be administered neat. In other embodiments, VEGI-192A and a pharmaceutically acceptable excipient are administered, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

Generally, these agents are formulated for administration by injection (e.g., intraperitoneally, intravenously, subcutaneously, intratumorally, intramuscularly, etc.), although other forms of administration (e.g., oral, mucosal, etc) can be also used. Administration can be systemic, e.g., intravenous and intraperitoneal, or localized. VEGI-192A protein is administered via site-specific or targeted local delivery techniques. Examples of site-specific or targeted local delivery techniques include various implantable depot sources of the VEGI-192A protein or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568. Accordingly, VEGI-192A protein are preferably combined with pharmaceutically acceptable vehicles such as saline, Ringer's solution, dextrose solution, and the like.

The particular dosage regimen, i.e., dose, timing and repetition, will depend on the particular individual and that individual's medical history. Generally, any of the following doses may be used: a dose of at least about 50 mg/kg body weight; at least about 20 mg/kg body weight; at least about 10 mg/kg body weight; at least about 5 mg/kg body weight; at least about 3 mg/kg body weight; at least about 1 mg/kg body weight; at least about 750 μg/kg body weight; at least about 500 μg/kg body weight; at least about 250 ug/kg body weight; at least about 100 μg/kg body weight; at least about 50 μg/kg body weight; at least about 10 ug/kg body weight; at least about 1 μg/kg body weight, or more, is administered. Empirical considerations, such as the half-life, generally will contribute to determination of the dosage.

In some individuals, more than one dose may be required. Frequency of administration may be determined and adjusted over the course of therapy, and is based on reducing the number of cancerous cells, maintaining the reduction of cancerous cells, reducing the growth and/or proliferation of cancerous cells, or delaying the development of metastasis. The presence of cancerous cells can be identified by any number of methods known to one of skill in the art or discussed herein (e.g., detection by immunohistochemistry or flow cytometry of biopsies or biological samples). In some cases, sustained continuous release formulations of VEGI-192A protein may be appropriate. Various formulations and devices for achieving sustained release are known in the art.

In one embodiment, dosages for VEGI-192A may be determined empirically in individuals who have been given one or more administration(s). Individuals are given incremental dosages of VEGI-192A. To assess efficacy of VEGI-192A, markers of the specific cancer disease state can be monitored. These markers include: direct measurements of tumor size via palpation or visual observation; indirect measurement of tumor size by x-ray or other imaging techniques; an improvement as assessed by direct tumor biopsy and microscopic examination of the tumor sample; the measurement of an indirect tumor marker (e.g., PSA for prostate cancer), a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of cancer, the stage of cancer, whether the cancer has begun to metastasize to other location in the individual, and the past and concurrent treatments being used.

Other formulations include suitable delivery forms known in the art including, but not limited to, carriers such as liposomes. See, for example, Mahato et al. (1997) Pharm. Res. 14:853-859. Liposomal preparations include, but are not limited to, cytofectins, multilamellar vesicles and unilamellar vesicles.

VEGI-192A may be used in conjunction with other cancer therapies, for example, radiation therapies or chemotherapeutic agents. VEGI-192A may be administered in conjunction with other cancer therapeutic agents, such as Rituxan® and Herceptin®.

Assessment of disease is performed using standard methods in the arts, such as imaging methods and monitoring appropriate marker(s).

IV. Compositions and Methods of Making the Compositions

The compositions used in the methods of the invention comprise a VEGI-192A protein. In some embodiments, the VEGI-192A protein is produced recombinantly. In some embodiments, the VEGI-192A protein is produced recombinantly from E. coli and purified. It is understood that the compositions can comprise a VEGI-192A and one or more other anti-cancer agents.

VEGI-192A (used interchangeably with VEGI-192A protein and VEGI-192A polypeptide) includes any naturally occurring species (such as full length from human or other mammalians), biologically active peptide fragments (e.g., VEGI-192A fragment described in WO 03/039491 and U.S. Pat. Pub. No. 2003/017242, and N-terminal truncated VEGI-192A fragment comprising amino acid 26 of SEQ ID NO: 1), and variants (including naturally occurring and non-naturally occurring), including functionally equivalent variants which do not significantly affect their biological properties and variants which have enhanced or decreased activity (e.g., inhibiting endothelian cell growth).

Nucleotide sequence and amino acid sequences of human VEGI-192A are described in PCT WO03/039491, U.S. Pat. Appl. Pub. No. 2003/0170242, and amino acid sequence of human VEGI-192A is also shown in Table 1 (SEQ ID NO:1). In some embodiments, VEGI-192A protein comprises amino acid sequence of SEQ ID NO: 1 shown in Table 1. VEGI-192A embodiments include fusion proteins (N-terminal fusion or C-terminal fusion), for example, N-terminal fusion protein shown in Table 2. VEGI-192A may be from any species, such as human.

TABLE 1
Amino acid sequence of human VEGI-192A

| | |
|---|---|
| Protein sequence of VEGI-192A | |
| MQLTKGRLHFSHPLSHTKHISPFVTDAPLRADGDKPR | (SEQ ID NO:1) |
| AHLTVVRQTPTQHFKNQFPALHWEHELGLAFTKNRMN | |
| YTNKFLLIPESGDYFIYSQVTFRGMTSECSEIRQAGR | |
| PNKPDSITVVITKVTDSYPEPTQLLMGTKSVCEVGSN | |
| WFQPIYLGAMFSLQEGDKLMVNVSDISLVDYTKEDKT | |
| FFGAFLL | |

The invention encompass methods using functionally equivalent variants of VEGI-192A. VEGI-192A and functionally equivalent variants of VEGI-192A of the invention are identified and characterized by any (one or more) of the following criteria: (a) ability to inhibit endothelial cell growth and/or proliferation; b) ability to induce endothelial cell death; b) ability to inhibit angiogenesis; c) ability to inhibit tumor growth (e.g., breast and lung cancer); d) ability to activate host immune system, for example ability to induce production of one or more cytokines (such as IL-15 and IP-10). Biological activity of variants of VEGI-192A may be tested using methods known in the art and methods described in Examples 2-5. In some embodiments, functionally equivalent variants have at least about any of 50%, 60%, 70%, 75%, 80%, 85%, 90%, or 95% of activity as compared to full length native VEGI-192A with respect to one or more of the biological assays described above (or known in the art). In some embodiments, functionally equivalent variants have an IC₅₀ of at least about any of 50%, 60%, 70%, 75%, 80%, 85%, 90%, or 95% of any of 1000 ng/ml, 100 ng/ml, 60 ng/ml, 40 ng/ml, 20 ng/ml, 12 ng/ml, or 6 ng/ml in inhibiting vascular endothelial cell proliferation in vitro (e.g., assay described in Example 2).

Variants of VEGI-192A of the present invention may include polypeptides which are at least about any of 70%, 75%, 80%, 85%, 90%, 95%, 96%, 97%, 98%, or 99% identical to VEGI-192A (e.g., SEQ ID NO:1).

Two polypeptide sequences are said to be “identical” if the sequence of amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e. the window size) and multiplying the results by 100 to yield the percentage of sequence identity.

Variants of VEGI-192A of the present invention may include one or more amino acid substitutions, deletions or additions that do not significantly change the activity of the protein. Variants may be from natural mutations or human manipulation. Changes can be of a minor nature, such as conservative amino acid substitutions that do not significantly affect the folding or activity of the protein. To improve or alter the characteristics of VEGI polypeptides, protein engineering may be employed. Recombinant DNA technology known to those skilled in the art can be used to create novel mutant proteins or mutants including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions. Thus, the invention also encompasses VEGI-192A derivatives and analogs that have one or more amino acid residues deleted, added, or substituted to generate VEGI-192A polypeptides that are better suited for expression, scale up, etc., in the host cells chosen.

Functionally equivalent variants of VEGI-192A used in the methods of the invention also encompass fusion proteins comprising VEGI-192A polypeptides. Biologically active VEGI-192A polypeptides can be fused with sequences, such as sequences that enhance immunological reactivity, facilitate the coupling of the polypeptide to a support or a carrier, or facilitate refolding and/or purification (e.g., sequences encoding epitopes such as Myc, HA derived from influenza virus hemagglutinin, His-6, FLAG, or the His-Tag shown in Table 2). These sequences may be fused to VEGI-192A polypeptide at the N-terminal end or at the C-terminal end. In addition, the protein or polynucleotide can be fused to other or polypeptides which increase its function, or specify its localization in the cell, such as a secretion sequence. Methods for producing recombinant fusion proteins described above are known in the art. The recombinant fusion protein can be produced, refolded and isolated by methods well known in the art. In some embodiments, the VEGI-192A protein used a fusion polypeptide comprising histidine residues, which may be prepared as described in the Examples. In some embodiments, the histidine fusion protein comprises SEQ ID NO:1.

It is understood that any of the VEGI-192A protein embodiments described herein do not include VEGI-174, VEGI-251, VEGI-192B. See U.S. Pat. Appl. Pub. No. 20020111325; U.S. Pat. Appl. Pub. No. 20030170242.

The composition used in the present invention can further comprise pharmaceutically acceptable carriers, excipients, or stabilizers (Remington: The Science and practice of Pharmacy 20th Ed. (2000) Lippincott Williams and Wilkins, Ed. K. E. Hoover.), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

VEGI-192A protein and compositions thereof can also be used in conjunction with other anti-cancer agents that serve to enhance and/or complement the effectiveness of the agents.

VEGI-192A (including variants) can be made using methods known in the art, for example, made recombinantly. For VEGI-192A and variants may be expressed in E. coli and refolded and purified according to methods described in U.S. Pat. No. 6,583,268, co-pending application (attorney docket no. 54411-20004.00, claiming priority to U.S. provisional application 60/528,983) and method described in the Examples.

V. Kits Comprising VEGI-192A for Therapeutic Purposes

The invention also provides kits for use in the instant methods. Kits of the invention include one or more containers comprising a purified VEGI-192A protein and instructions for use in accordance with any of the methods of the invention described herein. Generally, these instructions comprise a description of administration of a VEGI-192A protein to treat cancer (e.g., lung and breast cancer) according to any of the methods described herein. The kit may further comprise a description of selecting an individual suitable for treatment based on identifying whether that individual has cancer and the stage of the cancer.

The instructions relating to the use of VEGI-192A protein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating cancer (including metastatic cancer). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is VEGI-192A protein. The container may further comprise a second pharmaceutically active agent.

Kits may optionally provide additional components such as buffers and interpretive information. Normally, the kit comprises a container and a label or package insert(s) on or associated with the container.

In some embodiments, the invention provides articles of manufacture comprising contents of the kits described above. In some embodiments, the kits comprise a VEGI-192A protein with information indicating use to treat cancer (e.g., lung and breast cancer). In some embodiments, the kits comprise a VEGI-192A protein and another anti-cancer agent with information indicating use to treat cancer (e.g., lung and breast cancer) in conjunction with each other.

### EXAMPLES

### Example 1

### Refolding and Purification of Recombinant VEGI-192A

Vector construction and expression: A DNA fragment encoding human VEGI-192A as shown in Table 1 was produced by PCR amplification. The PCR product was inserted into the Nde I/Bam HI sites of pET-19b (Cat. No. 69677-3, Novagen, San Diego, Calif.), producing a VEGI-192A protein with a N-terminal fusion tag (Table 2). After PCR, ligation, and transformation into the BL21 DE3 strain of E. coli, single colonies were selected and amplified and then ultimately the construct was sequenced to assure the correctness of the DNA sequence. The nucleotide sequence and amino acid sequence of VEGI-192A with a N-terminal His-Tag are shown in Table 2.

TABLE 2

Nucleotide sequence and amino acid sequence
of VEGI-192A with a N-terminal His-Tag

| | |
|---|---|
| Gene coding sequence of VEGI-192A | |
| with a N-terminal His-Tag: | |
| ATGGGCCATCATCATCATCATCATCATCATCATCACA | (SEQ ID NO:2) |
| GCAGCGGCCATATCGACGACGACGACAAGCATATGCA | |
| ACTCACAAAGGGCCGTCTTCATTTCAGTCACCCTTTG | |
| TCTCATACAAAGCACATTTCTCCTTTTGTTACAGATG | |
| CACCTCTTAGAGCAGACGGAGATAAGCCAAGGGCACA | |
| CCTGACAGTTGTGAGACAAACTCCCACACAGCACTTT | |
| AAAAATCAGTTCCCAGCTCTGCACTGGGAACATGAAC | |
| TAGGCCTGGCCTTCACCAAGAACCGAATGAACTATAC | |
| CAACAAATTCCTGCTGATCCCAGAGTCGGGAGACTAC | |
| TTCATTTACTCCCAGGTCACATTCCGTGGGATGACCT | |
| CTGAGTGCAGTGAAATCAGACAAGCAGGCCGACCAAA | |
| CAAGCCAGACTCCATCACTGTGGTCATCACCAAGGTA | |
| ACAGACAGCTACCCTGAGCCAACCCAGCTCCTCATGG | |
| GGACCAAGTCTGTATGCGAAGTAGGTAGCAACTGGTT | |
| CCAGCCCATCTACCTCGGAGCCATGTTCTCCTTGCAA | |
| GAAGGGGACAAGCTAATGGTGAACGTCAGTGACATCT | |
| CTTTGGTGGATTACACAAAAGAAGATAAAACCTTCTT | |
| TGGAGCCTTCTTACTATAG | |
| Protein sequence of VEGI-192A | |
| with a N-terminal His-Tag: | |
| MGHHHHHHHHHHSSGHIDDDDKHMQLTKGRLHFSHPL | (SEQ ID NO:3) |
| SHTKHISPFVTDAPLRADGDKPRAHLTVVRQTPTQHF | |
| KNQFPALHWEHELGLAFTKNRMNYTNKFLLIPESGDY | |
| FIYSQVTFRGMTSECSEIRQAGRPNKPDSITVVITKV | |
| TDSYPEPTQLLMGTKSVCEVGSNWFQPIYLGAMFSLQ | |
| EGDKLMVNVSDISLVDYTKEDKTFFGAFLL | |

To produce VEGI-192A protein, VEGI-192A expression vector was transfected into BL21 DE3 strain of E. coli and plated on ZB plates with ampicillin. A single colony was selected and used to inoculate 100 mL of ZB media (10 g/l NZ amine A (Sigma) and 5 g/l NaCl) with ampicillin and grown overnight (approximately 16 hours) at 30° C. The 20 mL of the 100 mL starter culture was then used to inoculate 1 L of LB media with ampicillin, and the culture was incubated at 37° C. with shaking until the optical density at 600 nm (OD₆₀₀) reached 0.4-0.6. Isopropyl-beta-D-thiogalactopyranoside (IPTG) was then added to 0.5 mM to induce VEGI-192A expression, and the culture was incubated a further three hours with shaking. Large scale expression was accomplished utilizing multiple IL shaker flasks at 37° C.

Processing of Inclusion Bodies Before Refolding: The inclusion bodies were harvested from bacterial. Bacterial cells were collected by centrifugation, then resuspended in 20 mL of TN (150 mM NaCl, 50 mM tris, pH 8.0) with 1% TRITON X-100®. Ten milligrams of lysosyme was added, and the cell suspension was frozen at −20° C. overnight. The lysate was then thawed and 20 μL of 1 M magnesium sulfate and 100 μg of DNase were added. The cells were incubated, with stirring, until the released bacterial DNA was completely dissolved. The lysate was then diluted with 250 mL of TN with 1% TRITON X-100® and the mixture was stirred for 2-4 hours. Inclusion bodies were collected by centrifugation, and washed three times (by resuspension and centrifugation).

Refolding and Chromatographic Isolation of Refolded Protein: The washed inclusion bodies were dissolved in 8M urea, 0.1 M Tris, 1 mM glycine, 10 mM beta-mercaptoethanol, 10 mM dithiothreitol (DTT), 1 mM reduced glutathion (GSH), 0.1 mM oxidized glutathion (GSSG), pH 10.5. The absorbance at 280 nm (OD280) of the protein solution was adjusted to 2.0. The solution was clarified by ultracentrifugation (30 minutes×66,000g), then refolded.

The clarified solution was rapidly diluted into 20 volumes of 20 mM Tris, 1.36 mM Sodium Lauroyl Sarcosine, 0.009 mM Trimethylamine N-oxidedihydrate, 0.005 mM Cetyltrimethylammonium Bromide, pH 10.5. The resulting solution was adjusted to pH 8.0 with 1 M HCl stepwise over a 4-day period.

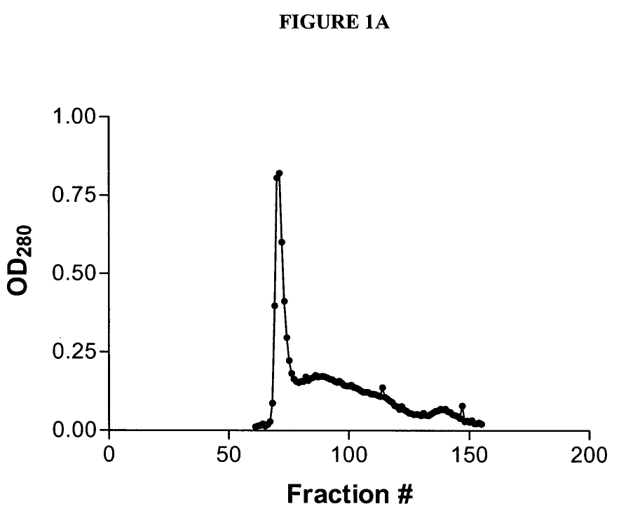

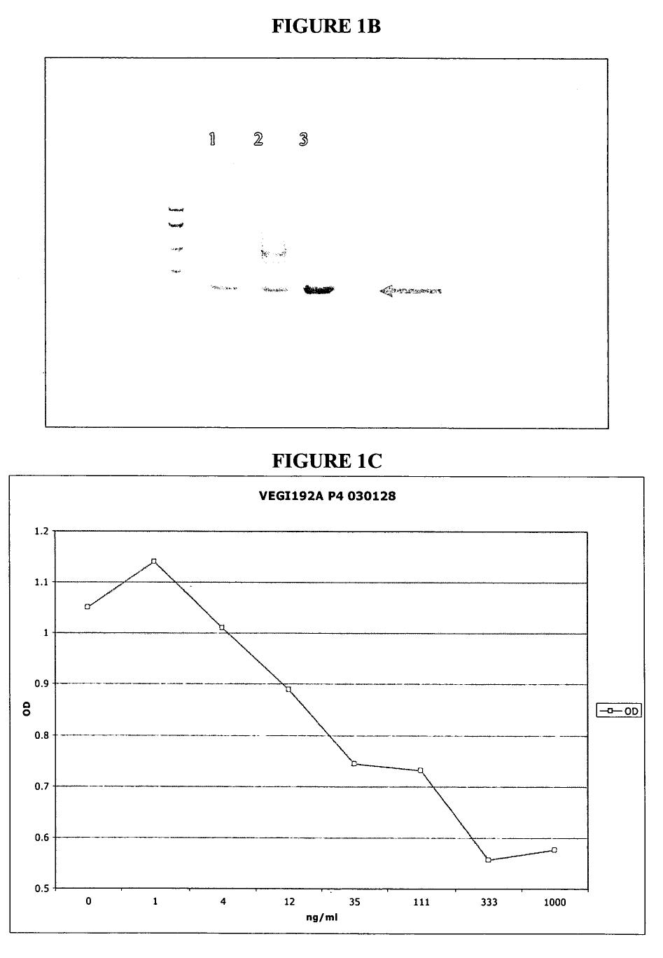

The refolded proteins were concentrated by ultrafiltration (Millipore Pellicon, 10,000 Da cut-off membrane and applied to a Sephacryl S-300 column equilibrated and eluted with 20 mM Tris, 0.4 M urea, 0.2 M NaCl, pH 8 (FIG. 1). Fractions from FIG. 1A were pooled (shown as 1, 2, 3) and concentrated to about 5 mg/ml (SDS-PAGE shown in FIG. 1B) for endothelial cell arrest assay and animal tumor model test described in Examples 2-4.

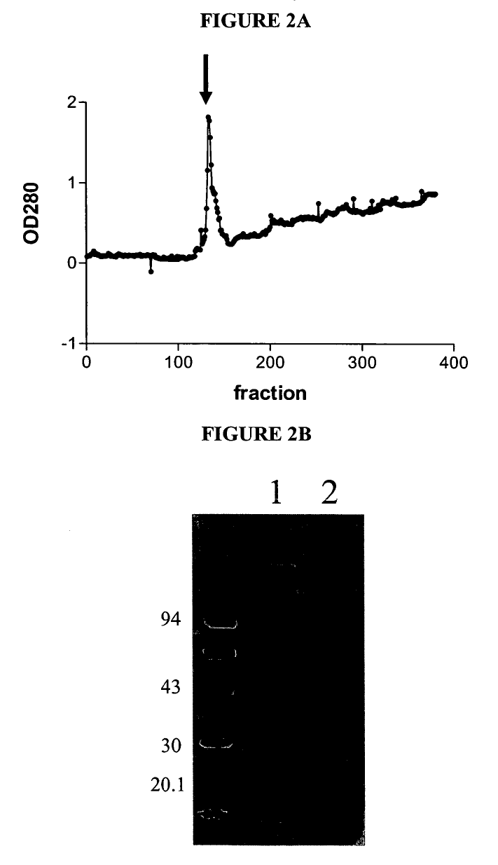

Functional VEGI-192A was also purified using Ni⁺-Affinity column. Ni⁺-chelating column (500 ml) was equilibrated with 20 mM Tris, 1.36 mM Sodium Lauroyl Sarcosine, 0.4 M urea, pH 8. Refolded VEGI-192A (4 L) was applied to the column, and the column was washed with 1 L of 20 mM Tris, 0.4 M urea, 0.2 M NaCl, 5 mM Immidozole, pH 8 (buffer A). VEGI-192A was eluted from the column with a linear gradient of immidozole (5 to 500 mM) in buffer A. The eluted peak shown in FIG. 2A was pooled, and dialyzed against 20 mM Tris, 0.4 M urea, 0.2 M NaCl, pH 8. The dialyzed VEGI-192A was then concentrated by ultrafiltration to about 5 mg/ml. The non-reduced and reduced SDS-PAGE analysis of produced VEGI-192A is shown in FIG. 2B.

In another experiment, the refolding process was performed by rapidly diluting the clarified solution containing the solubilized VEGI-192A into 20 volumes of 20 mM Tris, 0.034 mM Sodium Lauroyl Sarcosine, 0.009 mM Trimethylamine N-oxidedihydrate, 0.005 mM Cetyltrimethylammonium Bromide, pH 10.5. The resulting solution was adjusted to pH 8.0 with 1 M HCl stepwise over a 4-day period. The refolded VEGI-192A was concentrated and purified the same way as described above. The activity of the purified VEGI-192A using this refolding buffer was 100 times less than the refolding condition described above, determined by the endothelial cell arrest assay described in Example 2.

### Example 2

### Characterization of Biological Activity of Recombinant VEGI-192A by Endothelial Cell Growth Arrest Assay

Adult bovine aortic endothelial (ABAE) cells were cultured in IMEM (Gibco Biofluids, Rockville, Md.), 10% FBS, 1 ng/ml fibroblast growth factor-2 (Promega, Madison, Wis.), 37° C., 5% CO₂. The extent of quiescence of the cells was determined by ³H-thymidine incorporation. Cells were considered synchronized at G₀ phase of the cell cycle if no more than 5% of the cells were incorporating ³H-thymidine. The G₀-synchronized cells re-entered the growth cycle when they were re-seeded scarcely (5000 cells/cm2) in IMEM with 10% FBS and 1 ng/ml fibroblast growth factor-2, and incubated at 37° C., 5% CO₂. VEGI-192A prepared as described in Example 1 were added to the culture media either at the time of seeding the cells or at a time point when the cells entered the growth cycle in 20 hours post seeding. Single cell suspension was prepared from each culture well at a given time interval by trypsinization. The number of cells in each suspension was determined by using a Coulter counter, or by using a colorimetric assay utilizing a tetrazolium compound—MTS (Promega)—that can be metabolized by living cells to generate a blue colored compound detectable at 490 nm; the metabolic rate of MTS was proportional to the number of living cells in culture. Concentrations of VEGI-192A utilized in this assay ranged from 0.1 ng/mL to 11 g/mL.

In ABAE endothelial cell arrest assay using a colorimetric assay with MTS, all three pools were active with pool 3 being the most active (FIG. 1C). As shown in FIG. 1C, recombinant VEGI-192A in pool 3 exhibited an IC₅₀ of 12 ng/ml (0.49 nM) in inhibiting endothelial cell proliferation in vitro. In another experiment, recombinant VEGI-192A produced exhibited an IC₅₀ of 0.24 nM (6 ng/ml) in inhibiting endothelial cell proliferation in vitro.

### Example 3

### Inhibition of Lewis Lung Cancer Growth and Increase of Survival Time of the Tumor-Bearing Mice by Recombinant VEGI-192A

C57B16/J mice were implanted subcutaneously with Lewis lung carcinoma (LLC) cells. The carcinoma cells were washed with PBS, dispersed in a 0.05% solution of trypsin, and resuspended. After centrifugation at 4000 rpm for 10 min at 8° C., the cell pellet was resuspended in PBS and the concentration was adjusted to 2.5×10⁶ cells/ml. The mice were then injected subcutaneously on the flank with 0.1 ml of the carcinoma cell suspension. Tumors were measured with a dial-caliper, and tumor volumes were determined using the formula: Volume=width×width×length×0.52. After tumor volume was at least 100-200 mm³ (0.5-1% of body weight), which occurred within 5-7 days, mice were randomized into three groups. Two groups of mice received recombinant human VEGI-192A (prepared as described in Example 1) suspended in PBS at 5 mg/kg or 20 mg/kg, 2 times a week, by either intraperitoneal (IP) or intratumoral (IT) injection. IP or IT injection was started when the tumors were palpable, or when the tumor volumes reached 700-1000 mm³. The third group received comparable injections of the vehicle (phosphate-buffered saline) alone. The experiments were terminated and mice were sacrificed and autopsied when the tumor volumes in the control groups exceeded 2000 mm³.

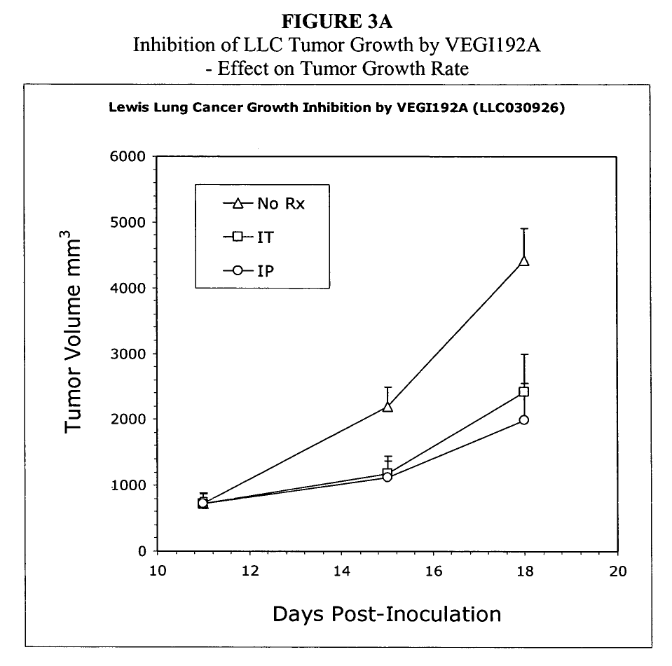

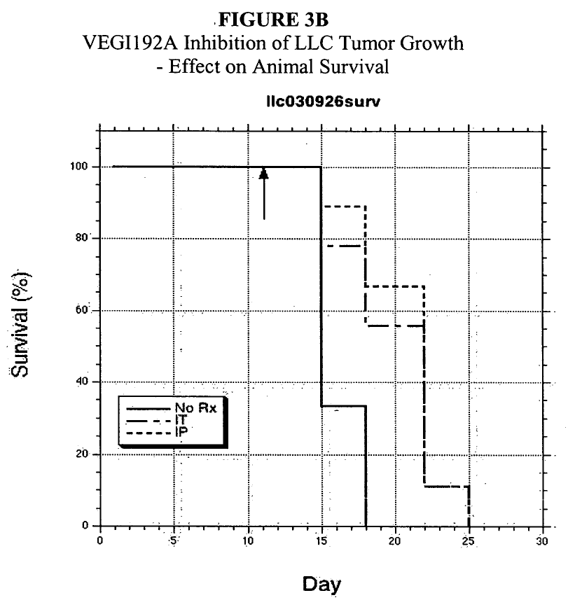

Substantial inhibition of the tumor growth rate in tumor-bearing animals was observed when recombinant VEGI-192A was delivered by intraperitoneal (IP) injection to the tumor-bearing animals when the tumors were palpable, or when the tumor volumes reached 700-1000 mm³. As shown FIG. 3A, systemic delivery of the recombinant VEGI-192A at 5 mg/Kg by IP or IT injection to mice with the tumor volumes of 700-1000 mm³ showed a marked inhibition of the tumor growth. As much as 50% inhibitions of the tumor growth rate was achieved even when the tumor volumes nearly 5% of the body weight (700-1000 mm³) at the time of the initiation of the treatment.

LLC tumors at the stage when the tumors have reached nearly 5% (the tumor volumes of 700-1000 mm³) of the body weight of the animals may resemble late stage human lung cancers in a clinical setting, as the animals with LLC tumors at this stage would have already developed widely spread micro-metastasis, particularly to the lung, which would grow into macro-metastasis once the primary tumors are removed surgically. See O'Reilly et al., Cell 79(2):315-28 (1994); Cao et al., J. Clin. Invest. 101(5): 1055-63 (1998).

The survival time of the tumor-bearing mice was also tested. The survival time was determined by the number of days between the day of implanting LLC cells and the day when the tumor volume reached beyond 3000 mm³, or when the animals became paralyzed due to tumor infiltration into the spine as the highly invasive tumors grew. The animals in the Lewis lung cancer model were sacrificed when the tumor volume reached beyond 3000 mm³, or when the animals became paralyzed due to tumor infiltration into the spine as the highly invasive tumors grew. As it is shown from a plot of the survival time and the number of animals survived at that time (FIG. 3B), the median survival time (starting from the time of treatment, which began on day 11 when the average sizes of the tumors was 700 mm³ for the untreated group) was about 4 days, whereas the median survival time for the treated group was 12 days, reflecting a 3× longer survival time.

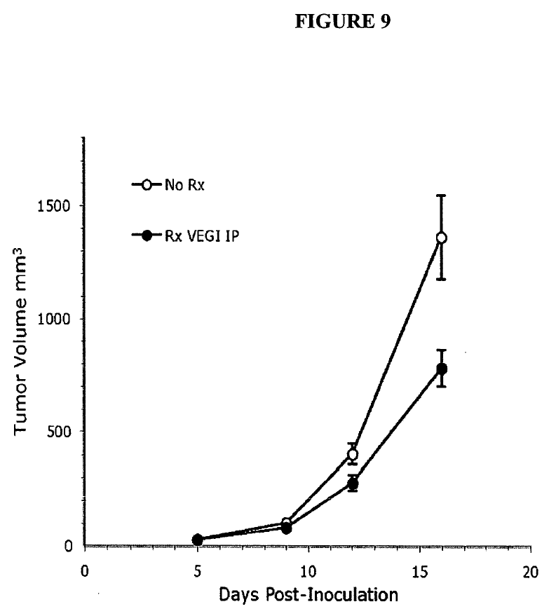

In another experiment, LLC cells (1×10⁶ per injection) were injected subcutaneously on the flank of C57BL/6 black mouse (Harlan, Indianapolis, Ind.) and the treatment was initiated at an early time when the tumors were palpable (FIG. 9). The animals were treated on day 5, day 9, and day 12 by IP administration of VEGI-192A (20 mg/kg). The control group was treated with vehicle. A significantly slower tumor growth rate was observed for the VEGI-treated group as shown in FIG. 9.

### Example 4

### Treating Human Breast Cancer Xenograft Tumor with Recombinant VEGI-192A

MDA-MB-231 human breast cancer cells were injected (1×10⁶ cells per injection) into the mammary fat pads of a female athymic nude mouse. The cancer cells injected formed a palpable tumor in 5-7 days. The tumor volume (V) was determined by using the equation V=0.52LW² for the volume of an oval. Once the tumor volumes reached about 100 mm³, the recombinant VEGI-192A produced as described in Example 1 was delivered to the animals (5 mg/Kg, two times a week) by subcutaneous (SC) injection at the tumor sites. The control groups were treated under identical experimental conditions except that vehicle (PBS) was used instead. The animals were sacrificed once the tumor volume exceeded 2000 mm³, or the tumor weight was more than 2 grams, which was about 10% of the body weight. Tumors, other organs (lung, liver, spleen), and peripheral blood were collected for bio-pathological analysis.

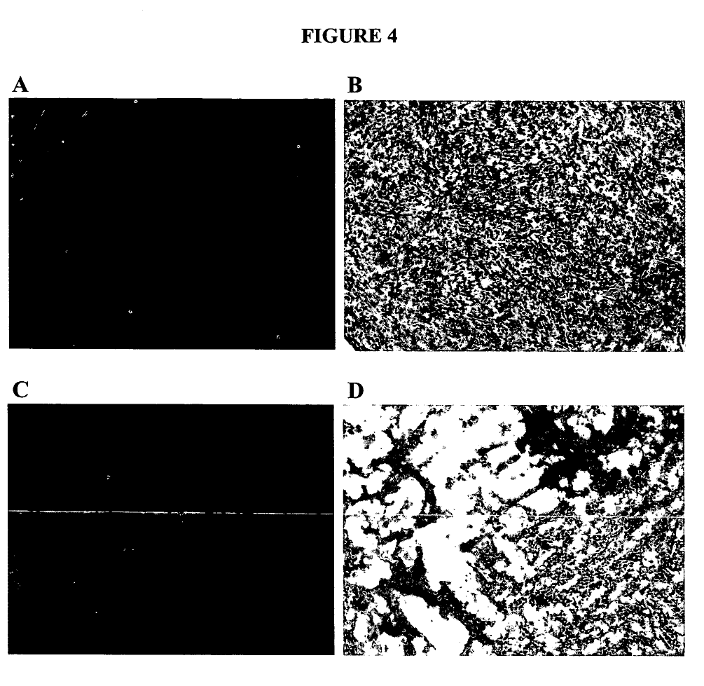

In order to analyze the effect of VEGI-192A treatment on the tumor vasculature, freshly frozen sections (from the animals of the control group and the group received SC injection at the tumor sites) were analyzed by fluorescent labeling of apoptotic cells (shown in Panels A and C of FIG. 4) or immunostaining for CD31, an endothelial cell marker (shown in Panels B and D of FIG. 4). In Panel A, untreated tumors showed little apoptotic cells and in Panel B untreated tumors showed abundant microvessels. In comparison, VEGI-192A-treated tumors (Panel C) showed intense fluorescent labeling of apoptotic cells and necrotic areas in tumor interior. As depicted in Panel D, VEGI-192A-treated tumors showed markedly decreased microvessel density. Cell death in tumor tissue was observed.

### Example 5

### Inhibition of In-Take of Lewis Line Cancer (LLC) Cells by Administration of Recombinant VEGI-192A

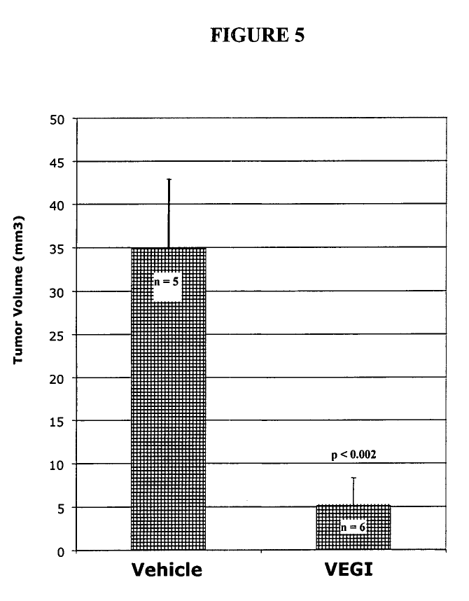

LLC cells were prepared as described in Example 3 and were inoculated (1×10⁵ cells/injection) on the back of C57B1 mice. The animals were treated by intraperitoneal (IP) injection of 5 mg/kg VEGI-192A (prepared as described in Example 1) at the same time of LLC cell inoculation (Day 0). Tumor volumes were determined on Day 4. As shown in FIG. 5, treatment of the animals at the time of tumor inoculation resulted in a marked inhibition of tumor in-take rate (t-test, p<0.002).

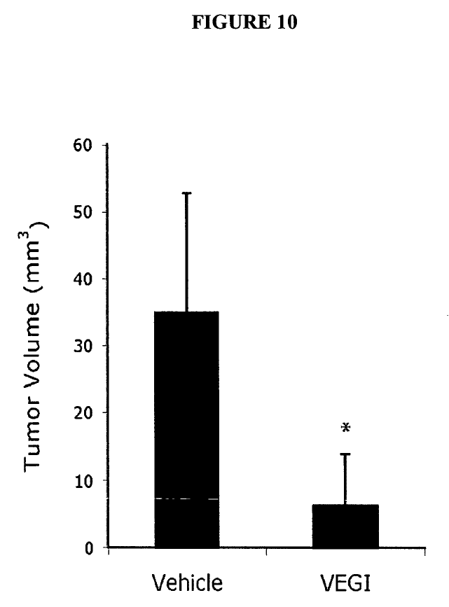

In another experiment (shown in FIG. 10), LLC cells (1×10⁶ per injection) were inoculated on the flank of C57BL/6 black mouse (Harlan, Indianapolis, Ind.) and the animals were treated with VEGI-192A (20 mg/kg, prepared as described in Example 1) immediately following cancer cell inoculation. The treatment was repeated daily until Day 4. When the tumor volumes were assessed on Day 5 post inoculation, all the animals in the vehicle-treated group had developed subcutaneous tumors (5/5 or 100%), with a mean volume (+/−SD) of 35 mm³, whereas about one-half of the VEGI-treated group exhibited measurable tumors (2/6 or 33%), and the tumor volumes were much smaller (FIG. 10). The results indicates that systemic administration of VEGI led to retardation of tumor formation by the cancer cells.

### Example 6

### Activation of the Host Immune System by Administration of Recombinant VEGI-192A

LLC cells were prepared as described in Example 3 and were inoculated (1×10⁵ cells/injection) on the back of C57B1 mice. Administration of VEGI-192A was initiated on Day 4 when the tumors became palpable. VEGI-192A (prepared as described in Example 1) was injected at 5 mg/Kg on Day 4, Day 7, Day 8, Day 9, and Day 10. Spleens were retrieved and weighed on Day 11. Sera were also collected on Day 11. Cytokine levels in sera were determined using an antibody-conjugated luminescent assay kit (LINCOplex, LINCO Research, Inc., Missouri) according to manufacture's instruction.

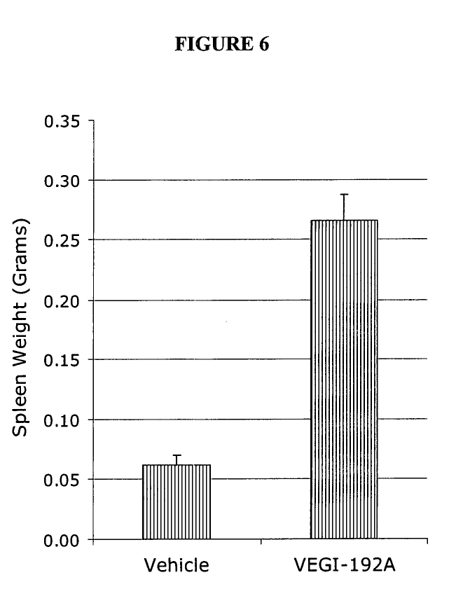

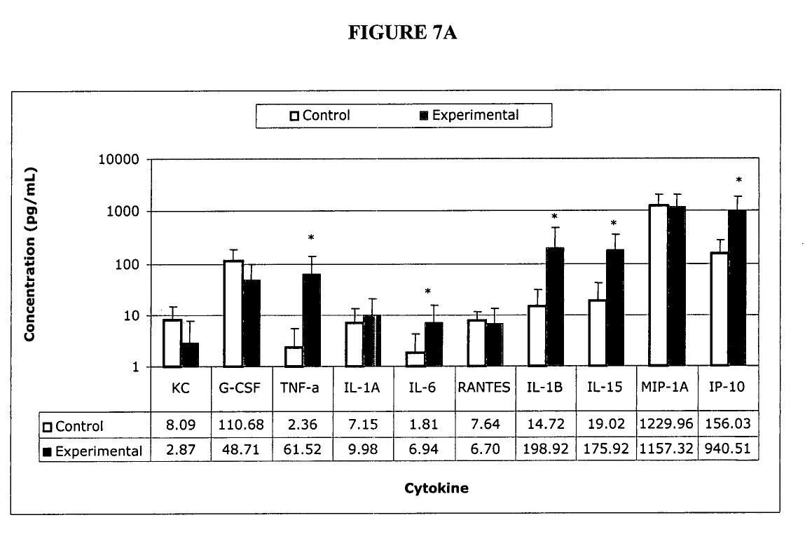

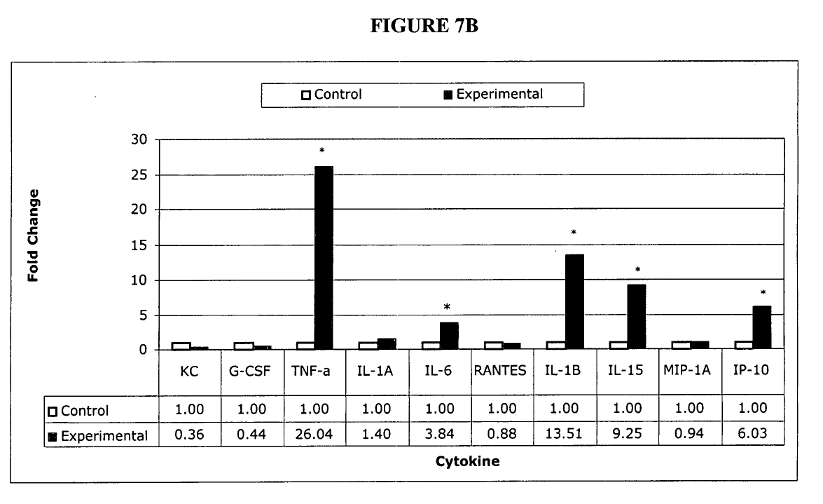

Treatment of the animals with VEGI-192A caused significantly enlarged spleens (shown in FIG. 6) and markedly enhanced production of several cytokines (shown in Figure 7), such as TNF-α, IL-6, IL-1B, IL-15, and IP-10. IL-15 and IP-10 are known to be involved in the activation of various lymphocytes and the inhibition of angiogenesis.

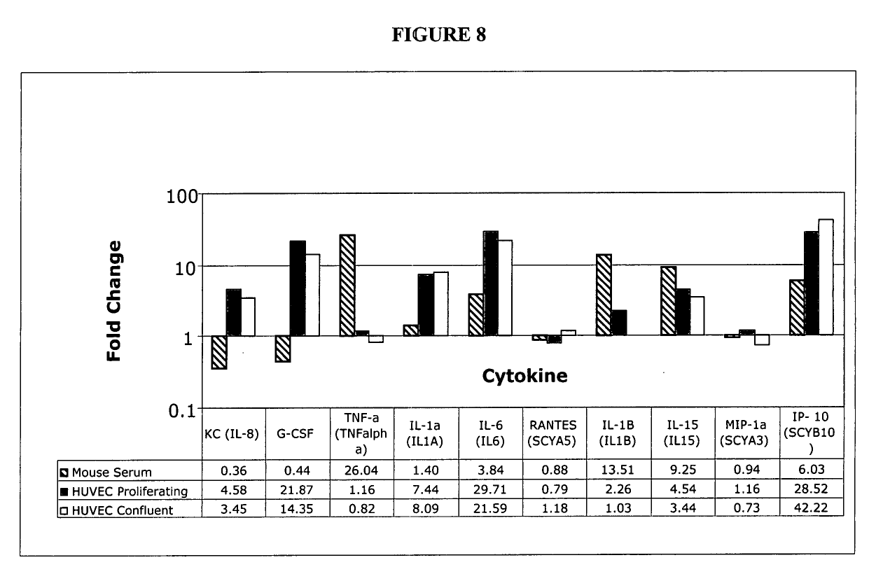

The above cytokine profiles in mouse serum was compared to cultured human endothelial cells in response to VEGI-192A treatment. Proliferating or confluent (G0-synchronized) human umbilical cord vein endothelial cells (HUVEC) were treated with 500 ng/ml VEGI-192A for five hours. After treatment, cytokine expression profiles for treated and untreated of both proliferating and confluent HUVEC were determined using a cDNA microarray obtained from Fred Hutchinson Cancer Research Center, Seattle, Wash. Cytokine profiles in mouse serum from FIG. 7B were compared to cytokine profiles for both proliferation and confluent HUVEC treated with VEGI-192A. As shown in FIG. 8, VEGI-192A treatment induced IL-15 and IP-10 expression in both proliferating and confluent HUVEC, suggesting that VEGI-192A treatment may enhance production of these two cytokines in human.

### Example 7

### Immunohistochemical Analysis of the Tumor Vasculature in LLC Tumor-Bearing Mice after VEGI-192A Treatment

We determined the impact of systemic treatment of the LLC tumor-bearing mice with VEGI-192A (prepared as described in Example 1) on the abundance and structure of the tumor blood vessels. LLC cells were prepared as described in Example 3 and were inoculated (1×10⁶ cells/injection) subcutaneously on the flank of C57BL/6 black mice (Harlan, Indianapolis, Ind.). Intraperitoneal administration of VEGI-192A or vehicle were initiated on Day 4. VEGI-192A (5 mg/Kg) was injected two times a week for three weeks. Vehicle-treated group received comparable injections of phosphate-buffered saline. Immunohistochemical analysis was performed as described below. Tumors were removed and placed into fixative with 4% paraformaldehyde in PBS at 4° C. for 4 hours, and transferred into 30% sucrose (4° C.) in RNase-free PBS overnight at 4° C. The tumors were then placed in OCT compound on dry ice. Sections (8 μm thickness) were dried on glass slides at 45° C. for two hours prior to immunostaining. Endothelial cells were identified with a rat monoclonal antibody to CD31 (PECAM-1; BD PharMingen, clone MEC 13.3, 1:250 dilution, cat.# 01951D). Vascular smooth muscle cells were identified with monoclonal anti-actin-α-FITC (Sigma, clone 1A4, 1:250 dilution, cat.# F3777). Vascular basement membrane was identified with a rabbit polyclonal antibody to type-IV collagen (1:10,000 dilution; Cosmo Bio Co., Tokyo, Japan). Nuclei were identified with Hoechst (100 ng/ml; Sigma). The secondary antibodies used were biotinylated anti-rat (Vector Laboratories, Burlingame, Calif. cat.#BA 4001), rabbit anti-rat IgG-TRITC (Sigma, cat. T4280), and donkey anti-rat IgG-FITC (Jackson cat.# 712-096-153), AMCA avidin D (Vector Laboratories, cat.# A-2008) for biotin detection, and goat anti-rabbit IgG-TRITC (Sigma cat. # T6778). ABC standard kit and DAB kit (Vector Laboratories) were used for the immunohistochemistry. The specimens were incubated with 0.03% H₂O₂ (Sigma) for 15 min at room temperature to block endogenous peroxidase activity, then with PBS containing 0.2% Triton X-100 and 5% bovine serum albumin (Sigma) to block nonspecific antibody binding. The specimens were then incubated with a primary antibody diluted to 1-2 μg/ml in PBS for 1 hour at room temperature, or 12-15 hours at 4° C. Control specimens were treated under identical conditions except that the primary antibody was replaced with PBS. After rinsing with PBS, the specimens were incubated with a secondary antibody for 1 hour at room temperature, rinsed with PBS, then incubated with ABC standard kit, or AMCA avidin D for 30 min at room temperature. The specimens were rinsed with PBS and incubated with DAB kit, then rinsed and mounted in Vectashield (Vector Laboratories).

The specimens were examined with a Nikon Eclipse E800 fluorescence microscope equipped with single, dual, and triple fluorescence filters and a low-light, RETIGA 1300C CCD Camera (Quantitative Imaging Corporation, Burnaby, BC, Canada) with Qcapture software. Images were saved as digital files. Image analysis were carried out with Image-pro plus software (Media Cybernetics, Inc) or Image-J (N1H).

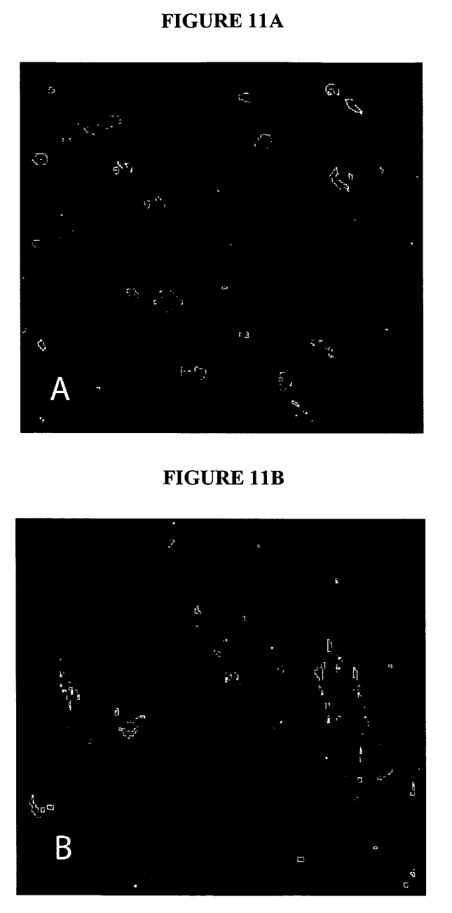

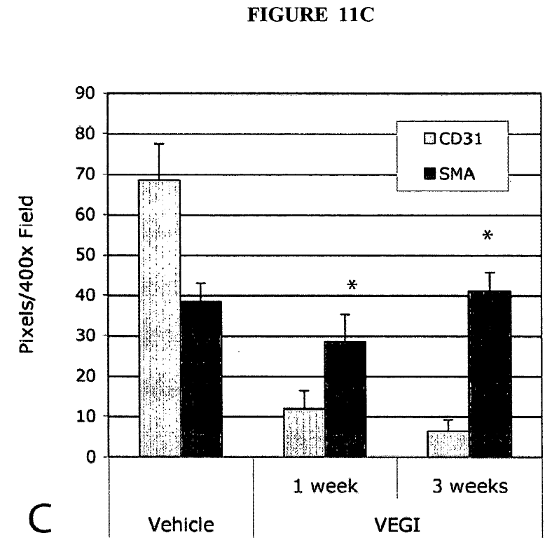

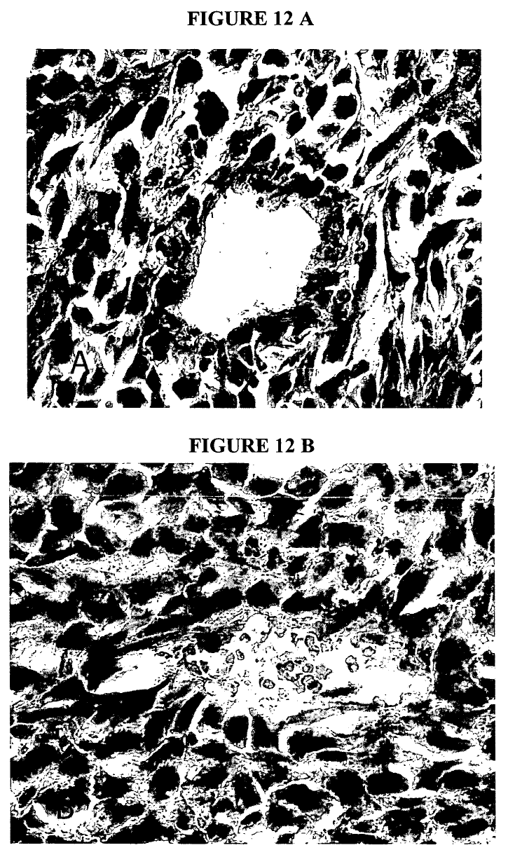

Specific eradication of endothelial cells in tumor: Freshly frozen tumors were sectioned and subjected to immunostaining for endothelial marker CD31 (red) and smooth muscle cell antigen-a (SMA-a) (green) (FIG. 11A, 11B). We then analyzed 15 fields on each slide that contained the most number of microvessels (“hot spots”) by computer-assisted image analysis. The densities of the red or green pixels per field (400× magnification) were determined (FIG. 11C). The results (as shown in FIG. 11) indicates a specific elimination of endothelial cells in tumor blood vessels. The density of the endothelial cells, measured as the total pixels occupied by CD31-positive cells, exhibited an 88% decrease within one week of treatment, and a further decrease within three weeks. Interestingly, the number of the smooth muscle cells remained relatively unchanged. As a result, the ratio of endothelial cells to smooth muscle cells decreased markedly in VEGI-treated tumors, changing from 1.8 to 0.4 and 1.8 to 0.15 after the animals had been treated for one or three weeks, respectively. A similar immunostaining was carried out for another endothelial cell marker, CD 105, and obtained identical results. These results indicate that the antiangiogenic activity of VEGI-191A caused specific eradication of tumor vascular endothelial cells.

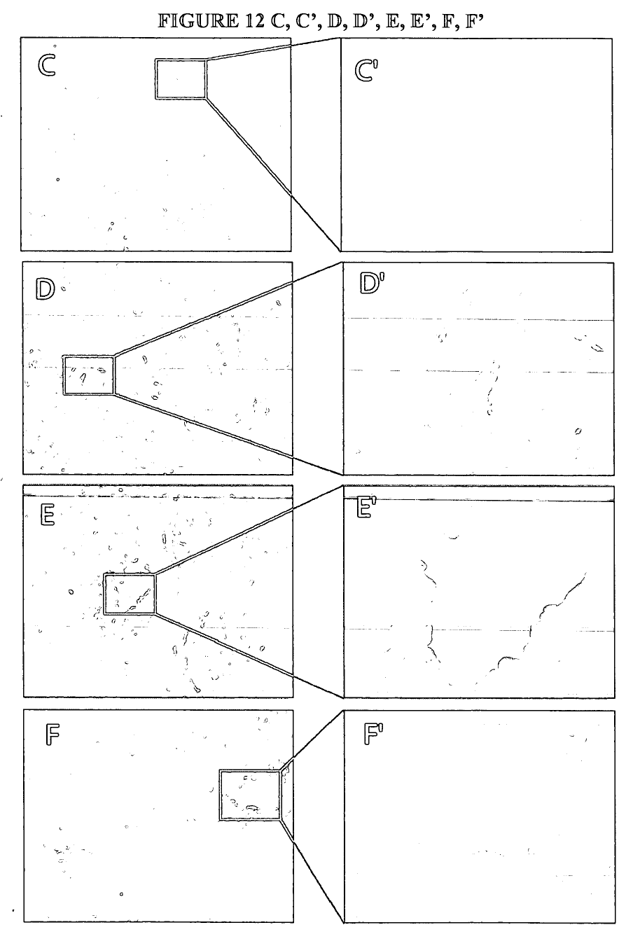

Persistence of vascular smooth muscle cells in tumors after elimination of endothelial cells: We found that vascular smooth muscle cells persisted for the duration of the experiment after the endothelial cells were eliminated (FIG. 12). The lumens of some of the tumor vasculature appeared to be somewhat maintained. This raised the possibility that the tumor blood vessels with good smooth muscle cell support may have retained some residual function after most of the endothelial cells were no longer present.

Presence of residual vascular structures in VEGI-192A-treated tumors: The residual vascular structure was further investigated. Unlike the lumens of the blood vessel in the vehicle-treated tumors which were always lined by endothelial cells (FIG. 12A), there were spaces in the VEGI-treated tumors that contained red blood cells but were not lined by endothelial cells (FIG. 12B). Whether these spaces were residual blood vessels that were depleted of endothelial cells was then determined. This was accomplished by immunostaining for collagen IV, a major component of the basement membrane of a blood vessel. As shown in FIGS. 12C and 12D, the basic structure of the tumor vasculature remained nearly intact even when the endothelial cells were nearly completely eliminated by VEGI-192A treatment (FIG. 12C), as compared with the readily detectable endothelial cells that were associated with the blood vessel basement membrane in the vehicle-treated tumors of the control group (FIG. 12D). These basement membrane structures were often accompanied by smooth muscle cells regardless whether the tumors were treated with VEGI-192A (FIG. 12E) or vehicle (FIG. 12F). The appearance of these residual vessels was not distinguishable from those seen in the untreated tumors, with the only difference that the vascular structures in the untreated tumors contain endothelial cells. These results indicate that a residual vascular structure consisting of basement membrane and smooth muscle cells exists, at least for the duration of the experiment, after endothelial cells are removed from these tumor vessels.

Although the foregoing invention has been described in some detail by way of illustration and example for purposes of clarity and understanding, it will be apparent to those skilled in the art that certain changes and modifications may be practiced. Therefore, descriptions and examples should not be construed as limiting the scope of the invention, which is delineated by the appended claims.

1. A method for treating lung cancer in an individual, comprising administering an effective amount of a VEGI-192A polypeptide.

2. The method of claim 1, wherein the cancer is advanced.

3. The method of claim 1, wherein the cancer is metastasized.

4. The method of claim 1, wherein the VEGI-192A polypeptide is human VEGI-192A.

5. The method of claim 1, wherein the VEGI-192A polypeptide comprises amino acid sequence of SEQ ID NO:1.

6. The method of claim 1, wherein the VEGI-192A polypeptide is produced recombinantly from E. coli.

7. A kit for treating lung cancer comprising a VEGI-192A polypeptide and an instruction for administering the VEGI-192A polypeptide to treat lung cancer.

8. A method for treating breast cancer in an individual, comprising administering an effective amount of a VEGI-192A polypeptide.

9. The method of claim 8, wherein the cancer is advanced.

10. The method of claim 8, wherein the cancer is metastasized.

11. The method of claim 8, wherein the VEGI-192A polypeptide is human VEGI-192A.

12. The method of claim 8, wherein the VEGI-192A polypeptide comprises amino acid sequence of SEQ ID NO:1.

13. The method of claim 8, wherein the VEGI-192A polypeptide is produced recombinantly from E. coli.

14. A kit for treating breast cancer comprising a VEGI-192A polypeptide and an instruction for administering the VEGI-192A polypeptide to treat breast cancer.


SEQUENCE LISTING (3 sequences)

SEQ ID NO: 1 - protein, 192 aa, Homo sapiens
MQLTKGRLHFSHPLSHTKHISPFVTDAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 2 - DNA, 648 bp, Artificial Sequence
  Synthesized VEGI-192A with a N-terminal His-Tag
atgggccatcatcatcatcatcatcatcatcatcacagcagcggccatatcgacgacgacgacaagcatatgcaactcacaaagggccgtcttcatttcagtcaccctttgtctcatacaaagcacatttctccttttgttacagatgcacctcttagagcagacggagataagccaagggcacacctgacagttgtgagacaaactcccacacagcactttaaaaatcagttcccagctctgcactgggaacatgaactaggcctggccttcaccaagaaccgaatgaactataccaacaaattcctgctgatcccagagtcgggagactacttcatttactcccaggtcacattccgtgggatgacctctgagtgcagtgaaatcagacaagcaggccgaccaaacaagccagactccatcactgtggtcatcaccaaggtaacagacagctaccctgagccaacccagctcctcatggggaccaagtctgtatgcgaagtaggtagcaactggttccagcccatctacctcggagccatgttctccttgcaagaaggggacaagctaatggtgaacgtcagtgacatctctttggtggattacacaaaagaagataaaaccttctttggagccttcttactatag

SEQ ID NO: 3 - protein, 215 aa, Artificial Sequence
  Synthesized VEGI-192A with a N-terminal His-Tag
MGHHHHHHHHHHSSGHIDDDDKHMQLTKGRLHFSHPLSHTKHISPFVTDAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL